(19) United States
(12) Patent Application Publication
(10) Pub. No.: US 2026/0294962 A1
(43) Pub. Date: Oct. 1, 2026

(54) COMPOSITIONS AND COMPOUNDS INCLUDING LITHIUM OROTATE

(71) Applicant: Atlantis Software Company, Houston, TX (US)

(72) Inventors: Elisa Medhus, Houston, TX (US); Rune Medhus, Houston, TX (US)

(21) Appl. No.: 19/563,229

(22) Filed: Mar. 11, 2026

Related U.S. Application Data
(60) Provisional application No. 63/780,685, filed on Mar. 31, 2025.

(51) Int. Cl. A61K 33/00; A61K 31/01; A61K 31/047; A61K 31/07; A61K 31/12; A61K 31/122; A61K 31/14; A61K 31/164; A61K 31/197; A61K 31/198; A61K 31/202; A61K 31/355; A61K 31/375; A61K 31/385; A61K 31/4188; A61K 31/4375; A61K 31/4415; A61K 31/455; A61K 31/51; A61K 31/519; A61K 31/525; A61K 31/593; A61K 31/714; A61K 33/04; A61K 33/06; A61K 33/10; A61K 33/18; A61K 33/20; A61K 33/24; A61K 33/26; A61K 33/30; A61K 33/32; A61K 33/34; A61K 33/42; A61K 36/53; A61K 36/67; A61K 36/81; A61K 36/88; A61K 36/9066; A61K 36/9068

(52) U.S. Cl. CPC A61K 33/00; A61K 31/01; A61K 31/047; A61K 31/07; A61K 31/12; A61K 31/122; A61K 31/14; A61K 31/164; A61K 31/197; A61K 31/198; A61K 31/202; A61K 31/355; A61K 31/375; A61K 31/385; A61K 31/4188; A61K 31/4375; A61K 31/4415; A61K 31/455; A61K 31/51; A61K 31/519; A61K 31/525; A61K 31/593; A61K 31/714; A61K 33/04; A61K 33/06; A61K 33/10; A61K 33/18; A61K 33/20; A61K 33/24; A61K 33/26; A61K 33/30; A61K 33/32; A61K 33/34; A61K 33/42; A61K 36/53; A61K 36/67; A61K 36/81; A61K 36/88; A61K 36/9066; A61K 36/9068

(57) ABSTRACT

Techniques are described for providing nutritional supplementation containing lithium compounds for cognitive and mental health support. For example, a composition may be configured to deliver lithium orotate in combination with vitamins, minerals, and botanical extracts in a capsule or other dosage form. The composition may utilize the lithium compound together with additional ingredients to support mood stabilization (e.g., through inclusion of L-theanine, magnesium, and saffron extract), provide antioxidant protection, and/or promote metabolic balance.

### CROSS-REFERENCE TO RELATED APPLICATION(S)

This application claims priority to U.S. Provisional Application No. 63/780,685 filed on Mar. 31, 2025 and entitled “COMPOSITIONS AND COMPOUNDS INCLUDING LITHIUM OROTATE,” which is incorporated herein by reference in its entirety.

### BACKGROUND

Many individuals suffer from cognitive disorders or conditions such as depression, mania, and bipolar disorders. Many patients of these cognitive disorders or conditions are often treated using antipsychotic medication and/or antidepressants in addition to, in some cases, anticonvulsants and/or anti-anxiety medications. Many of these medications have other side effects such as emotional blunting and an overall dulling of all types of emotions including emotions generally considered positive for an individual. Additionally, in some cases, a shortage of particular elements may potentially cause depression or other cognitive disorders or conditions even in otherwise fully healthy individuals and may significantly impact an individual's day to day functioning and overall quality of life.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 shows a method of treating a health condition through administration the compound including lithium orotate by medical professional.

FIGS. 2A-2H are an example of a pictorial diagram of various delivery methods for administration of the compound.

### DETAILED DESCRIPTION

Discussed herein are compositions and compounds including or containing lithium, particularly lithium orotate, that may in some cases (such as when recommended by a medical professional) be used to provide cognitive and/or mental health stabilization in individuals of various demographics, moods, and overall mental well-being. In some cases, the composition or compound including lithium orotate may be enclosed in a capsule. For example, the composition or compound may be formulated as oral dosage that may be taken or otherwise consumed on a periodic basis (e.g., daily, weekly, and/or the like). In some instances, the composition or compound including lithium orotate may be used as a mood stabilizer to assist individuals with the regulating their emotional and mental well-being.

For instance, in some cases, many individuals absorb or obtain many of elements required for positive health and well-being from their drinking water. However, in today's society, many individuals, particularly those with the urban environments, consume drinking water that's been purified or is otherwise been treated to remove harmful contaminants like bacteria, viruses, parasites, or other impurities to improve the overall safety of the water for the individual. Unfortunately, the purification treatment process often removes many of the trace elements otherwise and historically present in the drinking water of humans. In some cases, no substitute or alternative method of obtaining these trace elements exist in the typical 21st-century diet, thereby resulting in many individuals suffering from a shortage or deficiency of these trace elements that are typically beneficial to an individual's overall health and well-being, including lithium orotate.

Discussed herein, are compositions and compounds including lithium orotate may act as an alternative or supplement to the diet of individuals to thereby increase or stabilize the amount of trace elements in other dietary nutrients beneficial to the overall health and well-being of the individual. In some examples, the composition or compound may include lithium orotate as well as one or more of calcium, iodine, potassium iodide, magnesium, zinc, zinc citrate or zinc biglycinate, selenium, potassium, one or more omega-3 fatty acid (e.g., eicosapentaenoic acid (EPA), docosahexaenoic acid (DHA), and/or other the like), vitamin D3 (such as in the form of Cholecalciferol), vitamin B12 (such as in the form of Cyanocobalamin), vitamin A (such as in the form of Beta Carotene), vitamin E (such as in the form of Di-Alpha Tocopheryl Acetate), folate, and/or the like.

As another illustrative example, the composition or compound may include lithium orotate as well as one or more of vitamin D3 (such as in the form of Cholecalciferol), vitamin A (such as in the form of beta carotene), vitamin E (such as in the form of di-alpha tocopheryl Acetate), vitamin K2, vitamin C (such as in the form of ascorbic acid), thiamin, riboflavin, niacin, vitamin B6 (such as in the form of pyridoxine hydrochloride), folate, vitamin B12 (such as in the form of Cyanocobalamin), biotin, pantothenic acid (such as in the form of D-calcium pantothenate), calcium, phosphorus, iodine, potassium iodide, magnesium, zinc, zinc citrate or zinc biglycinate, selenium, copper, choline, manganese, chromium, chloride, potassium, lutein, lycopene, one or more omega-3 fatty acid (e.g., eicosapentaenoic acid (EPA), docosahexaenoic acid (DHA), and/or other the like), and/or the like.

As yet another illustrative example, the composition or compound may include lithium orotate as well as one or more of thiamin, riboflavin, niacin, vitamin B6 (such as in the form of pyridoxine hydrochloride), folate, vitamin B12 (such as in the form of Cyanocobalamin), one or more omega-3 fatty acid (e.g., eicosapentaenoic acid (EPA), docosahexaenoic acid (DHA), citicoline (e.g., cytidine-5′-diphosphocholine), Curcumin, L-Theanine, Coenzyme, Astaxanthin, Pterostilbene, vitamin D3 (such as in the form of cholecalciferol), Acetyl-Glutathione, alpha-glycerylphosphorylcholine, and/or the like.

As still another illustrative example, the composition or compound may include lithium orotate as well as one or more of thiamin, riboflavin, niacin, vitamin B6 (such as in the form of pyridoxine hydrochloride), folate, vitamin B12 (such as in the form of Cyanocobalamin), biotin, Pantothenic Acid (d-calcium pantothenate), vitamin D3 (such as in the form of Cholecalciferol), vitamin A (such as in the form of beta carotene), vitamin E (such as in the form of di-alpha tocopheryl Acetate), vitamin K2, vitamin C (such as in the form of ascorbic acid), calcium, phosphorus, iodine, potassium iodide, magnesium, zinc, zinc citrate or zinc biglycinate, iron, selenium, copper, manganese, chromium, chloride, potassium, lutein, one or more omega-3 fatty acid (e.g., eicosapentaenoic acid (EPA), docosahexaenoic acid (DHA), citicoline (e.g., cytidine-5′-diphosphocholine), and/or the like.

In various examples, the composition or compound provided above may also include one or more of the following, if not already included, saffron extract (or crocin), curcumin, theanine, Coenzyme Q10 (CoQ10), astaxanthin, pterostilbene, acetyl-Glutathione, alpha-glycerylphosphorylcholine, vitamin D3 (such as in the form of cholecalciferol), nicotinamide riboside, hydrogen malate (such as a precursor to nicotinamide adenine dinucleotide), L-taurine, R-lipoic acid, pyrroloquinoline quinone, superoxide dismutase, nicotinamide mononucleotide (NMN), quercetin, epigallocatechin-3-gallate (EGCG), sulforaphane, Kaempferol, fluoxetine, imipramine, citalopram, and/or the like. In some cases, the composition or compound may specifically exclude molybdenum.

In the various examples above, the quantity or amount of each element, nutritional additive, and/or mineral included in the composition or compound may differ and vary with respect to each other. For instance, the composition or compound may include between approximately 2.5 and 7.5 milligrams (mg) of lithium (such as in the form of lithium orotate), approximately 300 and 1400 mg of calcium, 50 and 200 micrograms (mcg) of iodine or potassium iodide, approximately 50 and 600 mg of magnesium, approximately 10 and 50 mg of zinc, zinc citrate, or zinc biglycinate, approximately 12.5 and 80 mcg of selenium, approximately 70 and 100 mg of potassium, approximately 800+800 and 250 mg of EPA/DHA (such as in a ratio of 2:1), approximately 5 and 30 mcg of vitamin D3 (such as approximately 400 and 1200 international units (IU)), approximately 1.4 and 3.4 mcg of vitamin B12, approximately 700 and 1100 mcg of vitamin A, approximately 50 and 150 mg of vitamin C, approximately 10 and 30 mg of vitamin E, approximately 200 and 500 mcg of vitamin B9 as L-methyl folate calcium or dietary folate equivalents.

As another example, the composition or compound may include between approximately 2.5 and 7.5 mg of lithium (such as in the form of lithium orotate), approximately 300 and 1400 mg of calcium, 50 and 200 mcg of iodine, approximately 50 and 600 mg of magnesium, approximately 5 and 16 mg of zinc, approximately 12.5 and 80 mcg of selenium, approximately 50 and 80 mg of potassium, approximately 250+250 and 1200+1200 mg of EPA/DHA (such as in a ratio of 2:1), approximately 5 and 30 mcg of vitamin D3 (such as approximately 400 and 1200 international units (IU)), approximately 1.4 and 3.4 mcg of vitamin B12, approximately 700 and 1100 mcg of vitamin A, approximately 50 and 150 mg of vitamin C, approximately 10 and 25 mg of vitamin E, approximately 200 and 500 mcg of vitamin B9 as L-methyl folate calcium or dietary folate equivalents.

As still another example, the composition or compound may include approximately 10 mg of lithium (such as in the form of lithium orotate), approximately 1200 mg of calcium, approximately 150 mcg of iodine (such as in the form of potassium iodide), approximately 420 mg of magnesium, approximately 11 mg of zinc (such as in the form of zinc citrate), approximately 55 mcg of selenium, approximately 3.5 g of potassium, approximately 1000+1000 mg of EPA/DHA (such as in a ratio of 2:1), approximately 20 mcg of vitamin D3 (such as approximately 800 IU), approximately 2.4 mcg of vitamin B12, approximately 900 mcg of vitamin A, approximately 90 mg of vitamin C, approximately 22.5 mg of vitamin E, and approximately 400 mcg of folate (such as approximately 667 mcg dietary folate equivalents).

As yet another example, the composition or compound may include between approximately 7.5 and 12.5 mg of lithium (such as in the form of lithium orotate), approximately 300 and 1400 mg of calcium, 50 and 200 mcg of iodine or potassium iodide, approximately 50 and 600 mg of magnesium, approximately 5 and 16 mg of zinc, approximately 12.5 and 80 mcg of selenium, approximately 50 and 100 mg of potassium, approximately 250+250 and 1200+1200 mg of EPA/DHA (such as in a ratio of 2:1), approximately 5 and 30 mcg of vitamin D3 (such as approximately 400 and 1200 international units (IU)), approximately 1.4 and 3.4 mcg of vitamin B12, approximately 700 and 1100 mcg of vitamin A, approximately 50 and 150 mg of vitamin C, approximately 10 and 25 mg of vitamin E, approximately 200 and 500 mcg of vitamin B9 as L-methyl folate calcium or dietary folate equivalents. In some cases, the increased lithium content may provide enhanced energy support for individuals requiring additional mood stabilization.

As a further example, the composition or compound may include between approximately 9 and 11 mg of lithium (such as in the form of lithium orotate), between approximately 1080 and 1320 mg of calcium, between approximately 135 and 165 mcg of iodine (such as in the form of potassium iodide), between approximately 378 and 462 mg of magnesium, between approximately 9.9 and 12.1 mg of zinc (such as in the form of zinc citrate), between approximately 49.5 and 60.5 mcg of selenium, between approximately 3.15 and 3.85 g of potassium, between approximately 900+900 and 1100+1100 mg of EPA/DHA (such as in a ratio of 2:1), between approximately 18 and 22 mcg of vitamin D3 (such as approximately 720 and 880 IU), between approximately 2.16 and 2.64 mcg of vitamin B12, between approximately 810 and 990 mcg of vitamin A, between approximately 81 and 99 mg of vitamin C, between approximately 20.25 and 24.75 mg of vitamin E, and between approximately 360 and 440 mcg of folate (such as approximately 600 and 734 mcg dietary folate equivalents).

As a further example, the composition or compound may include approximately 5 mg of lithium (such as in the form of lithium orotate), approximately 1200 mg of calcium, approximately 150 mcg of iodine (such as in the form of potassium iodide), approximately 420 mg of magnesium, approximately 11 mg of zinc (such as in the form of zinc citrate), approximately 55 mcg of selenium, approximately 3.5 g of potassium, approximately 1000+1000 mg of EPA/DHA (such as in a ratio of 2:1), approximately 20 mcg of vitamin D3 (such as approximately 800 IU), approximately 2.4 mcg of vitamin B12, approximately 900 mcg of vitamin A, approximately 90 mg of vitamin C, approximately 22.5 mg of vitamin E, and approximately 400 mcg of folate (such as approximately 667 mcg dietary folate equivalents).

As another example, the composition or compound may include between approximately 4.5 and 5.5 mg of lithium (such as in the form of lithium orotate), between approximately 1080 and 1320 mg of calcium, between approximately 135 and 165 mcg of iodine (such as in the form of potassium iodide), between approximately 378 and 462 mg of magnesium, between approximately 9.9 and 12.1 mg of zinc (such as in the form of zinc citrate), between approximately 49.5 and 60.5 mcg of selenium, between approximately 3.15 and 3.85 g of potassium, between approximately 900+900 and 1100+1100 mg of EPA/DHA (such as in a ratio of 2:1), between approximately 18 and 22 mcg of vitamin D3 (such as approximately 720 and 880 IU), between approximately 2.16 and 2.64 mcg of vitamin B12, between approximately 810 and 990 mcg of vitamin A, between approximately 81 and 99 mg of vitamin C, between approximately 20.25 and 24.75 mg of vitamin E, and between approximately 360 and 440 mcg of folate (such as approximately 600 and 734 mcg dietary folate equivalents).

As yet another example, the composition or compound may include between approximately 4 and 6 mg of lithium (such as in the form of lithium orotate), between approximately 960 and 1440 mg of calcium, between approximately 120 and 180 mcg of iodine (such as in the form of potassium iodide), between approximately 336 and 504 mg of magnesium, between approximately 8.8 and 13.2 mg of zinc (such as in the form of zinc citrate), between approximately 44 and 66 mcg of selenium, between approximately 2.8 and 4.2 g of potassium, between approximately 800+800 and 1200+1200 mg of EPA/DHA (such as in a ratio of 2:1), between approximately 16 and 24 mcg of vitamin D3 (such as approximately 640 and 960 IU), between approximately 1.92 and 2.88 mcg of vitamin B12, between approximately 720 and 1080 mcg of vitamin A, between approximately 72 and 108 mg of vitamin C, between approximately 18 and 27 mg of vitamin E, and between approximately 320 and 480 mcg of folate (such as approximately 534 and 800 mcg dietary folate equivalents).

As still another example, the composition or compound may include between approximately 3 and 7 mg of lithium (such as in the form of lithium orotate), between approximately 720 and 1680 mg of calcium, between approximately 90 and 210 mcg of iodine (such as in the form of potassium iodide), between approximately 252 and 588 mg of magnesium, between approximately 6.6 and 15.4 mg of zinc (such as in the form of zinc citrate), between approximately 33 and 77 mcg of selenium, between approximately 2.1 and 4.9 g of potassium, between approximately 600+600 and 1400+1400 mg of EPA/DHA (such as in a ratio of 2:1), between approximately 12 and 28 mcg of vitamin D3 (such as approximately 480 and 1120 IU), between approximately 1.44 and 3.36 mcg of vitamin B12, between approximately 540 and 1260 mcg of vitamin A, between approximately 54 and 126 mg of vitamin C, between approximately 13.5 and 31.5 mg of vitamin E, and between approximately 240 and 560 mcg of folate (such as approximately 400 and 934 mcg dietary folate equivalents).

As still another example, the composition or compound may include between approximately 2.5 and 7.5 mg of lithium (such as in the form of lithium orotate), approximately 300 and 1400 mg of calcium, 50 and 200 mcg of iodine, approximately 50 and 600 mg of magnesium, approximately 5 and 40 mg of zinc, approximately 12.5 and 80 mcg of selenium, approximately 80 and 100 mg of potassium, approximately 250 mg of EPA/DHA (such as in a ratio of 2:1), approximately 5 and 30 mcg of vitamin D3 (such as approximately 400 and 1200 IU), approximately 1.4 and 3.4 mcg of vitamin B12, approximately 700 and 1100 mcg of vitamin A, approximately 10 and 25 mg of vitamin E, approximately 60 and 110 mg of vitamin C, approximately 80 and 150 mcg of vitamin K2, approximately 0.5 and 2.1 mg of vitamin B6, approximately 0.5 and 2 mg of thiamin, approximately 0.5 and 2.1 mg of riboflavin, approximately 10 and 22 mg of niacin, approximately 10 and 50 mcg biotin, approximately 5 and 15 mg of pantothenic acid, approximately 500 and 900 mg of phosphorus, approximately 0.4 and 1.4 mg of copper, approximately 300 and 700 mg of choline, approximately 1.3 and 3.3 mg of manganese, approximately 25 and 45 mcg of chromium, approximately 1.3 and 3.3 g, chloride, approximately 5 and 20 mg of lutein, approximately 10 and 30 mg of lycopene, and approximately 200 and 600 mg of folate or dietary folate equivalents.

As still another example, the composition or compound may include between approximately 2.5 and 7.5 mg of lithium (such as in the form of lithium orotate), approximately 800+800 and 1200+1200 mg of EPA/DHA (such as in a ratio of 2:1), approximately 10 and 30 mcg of vitamin D3 (such as approximately 400 and 1200 IU), approximately 1.4 and 3.4 mcg of vitamin B12, approximately 80 and 150 mcg mcg of vitamin K2, approximately 1 and 3 mg of vitamin B6, approximately 0.5 and 2 mg of thiamin, approximately 1.3 and 2.2 mg of riboflavin, approximately 15 and 25 mg of niacin, approximately 100 and 500 mg of citicoline, approximately 600 and 1000 mcg of folate or folic acid, approximately 20 and 30 mg of Zinc, approximately 100 and 300 mg of curcumin, approximately 100 and 300 mg of L-Theanine, approximately 50 and 150 mg of coenzyme (such as coenzyme Q10), approximately 1 and 11 mg of astaxanthin, approximately 50 and 150 mg of pterostilbene, approximately 50 and 150 mg of acetyl-glutathione, and approximately 100 and 500 mg of alpha-glycerylphosphorylcholine.

As still another example, the composition or compound may include between approximately 2.5 and 7.5 mg of lithium (such as in the form of lithium orotate), approximately 800+800 and 1200+1200 mg of EPA/DHA (such as in a ratio of 2:1), approximately 10 and 30 mcg of vitamin D3 (such as approximately 400 and 1200 IU), approximately 1.4 and 3.4 mcg of vitamin B12, approximately 80 and 150 mcg of vitamin K2, approximately 1 and 3 mg of vitamin B6, approximately 0.5 and 2 mg of thiamin, approximately 1.3 and 2.2 mg of riboflavin, approximately 15 and 25 mg of niacin, approximately 100 and 500 mg of citicoline, approximately 600 and 1000 mcg of folate or folic acid, approximately 20 and 30 mg of zinc, approximately 100 and 300 mg of curcumin, approximately 100 and 300 mg of L-Theanine, approximately 50 and 150 mg of coenzyme Q10, approximately 1 and 11 mg of astaxanthin, approximately 50 and 150 mg of pterostilbene, approximately 50 and 150 mg of acetyl-glutathione, approximately 100 and 500 mg of alpha-glycerylphosphorylcholine, and approximately 30 and 88.5 mg of saffron extract. In some cases, the saffron extract may contain one or more of crocin, crocetin, safranal, picrocrocin, and kaempferol, which may provide antioxidant, mood stabilizing, and neuroprotective benefits.

As still another example, the composition or compound may include between approximately 2.5 and 7.5 mg of lithium (such as in the form of lithium orotate), approximately 800+800 and 1200+1200 mg of EPA/DHA (such as in a ratio of 2:1), approximately 10 and 30 mcg of vitamin D3 (such as approximately 400 and 1200 IU), approximately 1.4 and 3.4 mcg of vitamin B12, approximately 80 and 150 mcg of vitamin K2, approximately 1 and 3 mg of vitamin B6, approximately 0.5 and 2 mg of thiamin, approximately 1.3 and 2.2 mg of riboflavin, approximately 15 and 25 mg of niacin, approximately 100 and 500 mg of citicoline, approximately 600 and 1000 mcg of folate or folic acid, approximately 20 and 30 mg of zinc, approximately 100 and 300 mg of curcumin, approximately 100 and 300 mg of L-Theanine, approximately 50 and 150 mg of coenzyme Q10, approximately 1 and 11 mg of astaxanthin, approximately 50 and 150 mg of pterostilbene, approximately 50 and 150 mg of acetyl-glutathione, approximately 100 and 500 mg of alpha-glycerylphosphorylcholine, and approximately 20 and 100 mg of saffron extract. In some cases, the saffron extract may contain one or more of crocin, crocetin, safranal, picrocrocin, and kaempferol, which may provide antioxidant, mood stabilizing, and neuroprotective benefits.

As still another example, the composition or compound may include between approximately 2.5 and 7.5 mg of lithium (such as in the form of lithium orotate), approximately 1100 and 1500 mg of calcium, 80 and 170 mcg of iodine, approximately 190 and 290 mg of magnesium, approximately 3 and 13 mg of zinc, approximately 2 and 13 mg of iron, approximately 20 and 60 mcg of selenium, approximately 1.4 and 3.4 g of potassium, approximately 1800+1800 and 2200+2200 mg of EPA/DHA (such as in a ratio of 2:1), approximately 10 and 20 mcg of vitamin D3 (such as approximately 400 and 800 IU), approximately 1.3 and 2.2 mcg of vitamin B12, approximately 400 and 800 mcg of vitamin A, approximately 7 and 17 mg of vitamin E, approximately 50 and 100 mg of vitamin C, approximately 80 and 150 mcg of vitamin K2, approximately 0.5 and 1.5 mg of vitamin B6, approximately 0.4 and 1.4 mg of thiamin, approximately 0.4 and 1.4 mg of riboflavin, approximately 7 and 17 mg of niacin, approximately 10 and 30 mcg biotin, approximately 1 and 7 mg of pantothenic acid, approximately 1000 and 1500 mg of phosphorus, approximately 500 and 900 mcg of copper, approximately 1.3 and 2.3 mg of manganese, approximately 19 and 29 mcg of chromium, approximately 1.8 and 2.8 g chloride, approximately 1 and 11 mg of lutein, and approximately 100 and 500 mcg of folate or dietary folate equivalents.

In the various examples above, the quantity or amount of each element, nutritional additive, and/or mineral included in the composition or compound may differ and vary with respect to each other. For instance, the composition or compound may include between approximately 4.0 and 6.0 mg of lithium (such as in the form of 5.0 mg of lithium orotate), approximately 1150 and 1250 mg of calcium, 125 and 175 mcg of iodine or potassium iodide, approximately 400 and 450 mg of magnesium, approximately 20 and 40 mg of zinc, approximately 45 and 65 mcg of selenium, approximately 3.0 and 5.0 g of potassium, approximately 950+950 and 1050+1050 mg of EPA/DHA (such as in a ratio of 2:1), approximately 15 and 25 mcg of vitamin D3, approximately 2.0 and 3.0 mcg of vitamin B12, approximately 850 and 950 mcg of vitamin A, approximately 80 and 100 mg of vitamin C, approximately 20 and 25 mg of vitamin E, approximately 350 and 450 mg of folate or dietary folate equivalents.

As another example, the composition or compound may include between approximately 4.0 and 6.0 mg of lithium (such as in the form of 5.0 mg of lithium orotate), approximately 1150 and 1250 mg of calcium, 125 and 175 mcg of iodine, approximately 400 and 450 mg of magnesium, approximately 8 and 14 mg of zinc, approximately 45 and 65 mcg of selenium, approximately 3.0 and 5.0 g of potassium, approximately 950+950 and 1050+1050 mg of EPA/DHA (such as in a ratio of 2:1), approximately 15 and 25 mcg of vitamin D3 (such as approximately 400 and 1200 international units (IU)), approximately 2.0 and 3.0 mcg of vitamin B12, approximately 850 and 950 mcg of vitamin A, approximately 80 and 100 mg of vitamin C, approximately 20 and 25 mg of vitamin E, approximately 350 and 450 mg of folate or dietary folate equivalents.

As still another example, the composition or compound may include between approximately 4.0 and 6.0 mg of lithium (such as in the form of 5.0 mg of lithium orotate), approximately 1150 and 1250 mg of calcium, 125 and 175 mcg of iodine, approximately 400 and 450 mg of magnesium, approximately 5 and 14 mg of zinc, approximately 45 and 65 mcg of selenium, approximately 3.0 and 4.0 g of potassium, approximately 950+950 and 1050+1050 mg of EPA/DHA (such as in a ratio of 2:1), approximately 15 and 25 mcg of vitamin D3, approximately 12.0 and 3.0 mcg of vitamin B12, approximately 850 and 950 mcg of vitamin A, approximately 80 and 100 mg of vitamin C, approximately 20 and 25 mg of vitamin E, approximately 80 and 150 mcg of vitamin K2, approximately 1.0 and 1.6 mg of vitamin B6, approximately 1.0 and 1.5 mg of thiamin, approximately 1.0 and 1.6 mg of riboflavin, approximately 12 and 19 mg of niacin, approximately 25 and 35 mcg biotin, approximately 7.5 and 12.5 mg of pantothenic acid, approximately 650 and 750 mg of phosphorus, approximately 0.7 and 1.1 mg of copper, approximately 450 and 550 mg of choline, approximately 2.0 and 2.6 mg of manganese, approximately 30 and 40 mcg of chromium, approximately 2.0 and 2.6 g, chloride, approximately 12 and 18 mg of lutein, approximately 17 and 23 mg of lycopene, and approximately 350 and 450 mg of folate or dietary folate equivalents.

As still another example, the composition or compound may include between approximately 4.0 and 6.0 mg of lithium (such as in the form of 5.0 mg of lithium orotate), approximately 950+950 and 1050+1050 mg of EPA/DHA (such as in a ratio of 2:1), approximately 22 and 28 mcg of vitamin D3, approximately 2.0 and 3.0 mcg of vitamin B12, approximately 80 and 150 mcg of vitamin K2, approximately 1.5 and 2.5 mg of vitamin B6, approximately 1.0 and 2.0 mg of thiamin, approximately 1.5 and 2.0 mg of riboflavin, approximately 18 and 22 mg of niacin, approximately 250 and 350 mg of citicoline, approximately 750 and 850 mcg of folate or folic acid, approximately 25 and 25 mg of Zinc, approximately 150 and 250 mg of curcumin, approximately 150 and 250 mg of L-Theanine, approximately 75 and 125 mg of coenzyme, approximately 4 and 8 mg of astaxanthin, approximately 75 and 125 mg of pterostilbene, approximately 75 and 125 mg of acetyl-glutathione, and approximately 250 and 350 mg of alpha-glycerylphosphorylcholine.

As still another example, the composition or compound may include between approximately 4.0 and 6.0 mg of lithium (such as in the form 5.0 mg of lithium orotate), approximately 1200 and 1400 mg of calcium, 110 and 130 mcg of iodine, approximately 220 and 260 mg of magnesium, approximately 10 and 13 mg of zinc, approximately 5 and 10 mg of iron, approximately 35 and 45 mcg of selenium, approximately 2.0 and 2.8 g of potassium, approximately 1900+1900 and 2100+2100 mg of EPA/DHA (such as in a ratio of 2:1), approximately 12 and 18 mcg of vitamin D3, approximately 1.5 and 2.0 mcg of vitamin B12, approximately 550 and 650 mcg of vitamin A, approximately 10 and 15 mg of vitamin E, approximately 70 and 80 mg of vitamin C, approximately 55 and 65 mcg of vitamin K2, approximately 0.8 and 1.3 mg of vitamin B6, approximately 0.6 and 1.2 mg of thiamin, approximately 0.6 and 1.2 mg of riboflavin, approximately 10 and 15 mg of niacin, approximately 15 and 25 mcg biotin, approximately 2 and 6 mg of pantothenic acid, approximately 1150 and 1350 mg of phosphorus, approximately 650 and 750 mcg of copper, approximately 1.5 and 2.0 mg of manganese, approximately 21 and 27 mcg of chromium, approximately 2.0 and 2.5 g chloride, approximately 4 and 8 mg of lutein, and approximately 250 and 350 mcg of folate or dietary folate equivalents.

In some aspects, the composition or compound may include various ingredients in different ranges. For instance, the composition or compound may include between approximately 4.5 mg and 5.5 mg of lithium (elemental from orotate), between approximately 540 mg and 660 mg of palmitoylethanolamide (PEA), between approximately 450 mg and 550 mg of DL-phenylalanine (DLPA), between approximately 180 mg and 220 mg of L-theanine, between approximately 108 mg and 132 mg of magnesium (elemental from glycinate chelate), between approximately 270 mg and 330 mg of ashwagandha extract, between approximately 270 mg and 330 mg of dihydroberberine (DHB), between approximately 450 mg and 550 mg of myo-inositol, between approximately 180 mcg and 220 mcg of chromium (as chromium picolinate), between approximately 50 mg and 150 mg of microcrystalline cellulose, between approximately 25 mg and 75 mg of rice flour, between approximately 5 mg and 15 mg of silicon dioxide, and between approximately 5 mg and 20 mg of vegetable magnesium stearate.

In some cases, the composition or compound may include one or more additional ingredients beyond those specified in the examples above. For instance, the composition or compound may further include one or more of microcrystalline cellulose, rice flour, silicon dioxide, and vegetable magnesium stearate as excipients or flow agents to facilitate capsule manufacturing and stability. For instance, the composition or compound may include between approximately 4 mg and 6 mg of lithium (elemental from orotate), between approximately 480 mg and 720 mg of palmitoylethanolamide (PEA), between approximately 400 mg and 600 mg of DL-phenylalanine (DLPA), between approximately 160 mg and 240 mg of L-theanine, between approximately 96 mg and 144 mg of magnesium (elemental from glycinate chelate), between approximately 240 mg and 360 mg of ashwagandha extract, between approximately 240 mg and 360 mg of dihydroberberine (DHB), between approximately 400 mg and 600 mg of myo-inositol, between approximately 160 mcg and 240 mcg of chromium (as chromium picolinate), between approximately 75 mg and 200 mg of microcrystalline cellulose, between approximately 30 mg and 100 mg of rice flour, between approximately 8 mg and 25 mg of silicon dioxide, and between approximately 8 mg and 30 mg of vegetable magnesium stearate.

As yet another example, the composition or compound may include between approximately 3 mg and 7 mg of lithium (elemental from orotate), between approximately 360 mg and 840 mg of palmitoylethanolamide (PEA), between approximately 300 mg and 700 mg of DL-phenylalanine (DLPA), between approximately 120 mg and 280 mg of L-theanine, between approximately 72 mg and 168 mg of magnesium (elemental from glycinate chelate), between approximately 180 mg and 420 mg of ashwagandha extract, between approximately 180 mg and 420 mg of dihydroberberine (DHB), between approximately 300 mg and 700 mg of myo-inositol, between approximately 120 mcg and 280 mcg of chromium (as chromium picolinate), between approximately 40 mg and 250 mg of microcrystalline cellulose, between approximately 20 mg and 120 mg of rice flour, between approximately 3 mg and 30 mg of silicon dioxide, and between approximately 3 mg and 40 mg of vegetable magnesium stearate.

In some instances, palmitoylethanolamide (PEA) may support endocannabinoid system function and may in some aspects assist with modulating reward signaling pathways and reducing unwanted cravings. DL-phenylalanine (DLPA) may support dopamine synthesis and may in some cases assist with promoting dopamine balance and emotional steadiness. L-theanine may support calm focus and relaxation without sedation, which may in some aspects assist with impulse control and stress-related urges. Ashwagandha extract may support stress response through modulation of cortisol levels and may in some instances assist with emotional steadiness and managing stress-related urges.

In some aspects, the composition or compound may be formulated for premenstrual support, including monthly mood balance, cramp comfort, and healthy neurotransmitter and prostaglandin balance. For instance, the composition or compound may include approximately 5 mg of lithium (as lithium orotate, providing approximately 1 mg elemental lithium), approximately 200 mg of Vitex agnus-castus (chasteberry) extract, approximately 30 mg of saffron stigma extract, approximately 250 mg of ginger root extract, approximately 50 mg of curcumin extract, approximately 5 mg of black pepper fruit extract (piperine), approximately 200 mg of L-theanine, approximately 300 mg of magnesium bisglycinate chelate (providing approximately 60 mg elemental magnesium), approximately 20 mg of vitamin B6 (as pyridoxal-5-phosphate), and approximately 200 mg of myo-inositol. In some cases, the composition may be provided in a two-capsule daily serving format using size 00 capsules.

As one alternative example, the composition or compound may include between approximately 4.5 mg and 5.5 mg of lithium (as lithium orotate), between approximately 180 mg and 220 mg of Vitex agnus-castus extract, between approximately 27 mg and 33 mg of saffron stigma extract, between approximately 225 mg and 275 mg of ginger root extract, between approximately 45 mg and 55 mg of curcumin extract, between approximately 4.5 mg and 5.5 mg of black pepper fruit extract, between approximately 180 mg and 220 mg of L-theanine, between approximately 270 mg and 330 mg of magnesium bisglycinate chelate, between approximately 18 mg and 22 mg of vitamin B6, and between approximately 180 mg and 220 mg of myo-inositol.

As another example, the composition or compound may include between approximately 4 mg and 6 mg of lithium (as lithium orotate), between approximately 160 mg and 240 mg of Vitex agnus-castus extract, between approximately 24 mg and 36 mg of saffron stigma extract, between approximately 200 mg and 300 mg of ginger root extract, between approximately 40 mg and 60 mg of curcumin extract, between approximately 4 mg and 6 mg of black pepper fruit extract, between approximately 160 mg and 240 mg of L-theanine, between approximately 240 mg and 360 mg of magnesium bisglycinate chelate, between approximately 16 mg and 24 mg of vitamin B6, and between approximately 160 mg and 240 mg of myo-inositol.

As a further example, the composition or compound may include between approximately 3 mg and 7 mg of lithium (as lithium orotate), between approximately 120 mg and 280 mg of Vitex agnus-castus extract, between approximately 18 mg and 42 mg of saffron stigma extract, between approximately 150 mg and 350 mg of ginger root extract, between approximately 30 mg and 70 mg of curcumin extract, between approximately 3 mg and 7 mg of black pepper fruit extract, between approximately 120 mg and 280 mg of L-theanine, between approximately 180 mg and 420 mg of magnesium bisglycinate chelate, between approximately 12 mg and 28 mg of vitamin B6, and between approximately 120 mg and 280 mg of myo-inositol.

In some aspects, the composition or compound may be formulated for longevity support, including cellular health, antioxidant protection, and metabolic function. For instance, the composition or compound may include approximately 5 mg of lithium (as lithium orotate), approximately 100 mg of magnesium (as magnesium glycinate), approximately 8 mg of zinc (as zinc bisglycinate chelate), approximately 100 mcg of selenium (as selenomethionine), approximately 6 mg of astaxanthin, approximately 75 mg of coenzyme Q10 (ubiquinone), and approximately 100 mg of alpha-lipoic acid. In some cases, the composition may be provided in a two-capsule daily serving format.

As one alternative example, the composition or compound may include between approximately 4.5 mg and 5.5 mg of lithium (as lithium orotate), between approximately 90 mg and 110 mg of magnesium (as magnesium glycinate), between approximately 7.2 mg and 8.8 mg of zinc (as zinc bisglycinate chelate), between approximately 90 mcg and 110 mcg of selenium (as selenomethionine), between approximately 5.4 mg and 6.6 mg of astaxanthin, between approximately 67.5 mg and 82.5 mg of coenzyme Q10 (ubiquinone), and between approximately 90 mg and 110 mg of alpha-lipoic acid.

As another example, the composition or compound may include between approximately 4 mg and 6 mg of lithium (as lithium orotate), between approximately 80 mg and 120 mg of magnesium (as magnesium glycinate), between approximately 6.4 mg and 9.6 mg of zinc (as zinc bisglycinate chelate), between approximately 80 mcg and 120 mcg of selenium (as selenomethionine), between approximately 4.8 mg and 7.2 mg of astaxanthin, between approximately 60 mg and 90 mg of coenzyme Q10 (ubiquinone), and between approximately 80 mg and 120 mg of alpha-lipoic acid.

As a further example, the composition or compound may include between approximately 3 mg and 7 mg of lithium (as lithium orotate), between approximately 60 mg and 140 mg of magnesium (as magnesium glycinate), between approximately 4.8 mg and 11.2 mg of zinc (as zinc bisglycinate chelate), between approximately 60 mcg and 140 mcg of selenium (as selenomethionine), between approximately 3.6 mg and 8.4 mg of astaxanthin, between approximately 45 mg and 105 mg of coenzyme Q10 (ubiquinone), and between approximately 60 mg and 140 mg of alpha-lipoic acid.

As another example, the composition or compound may include approximately 5 mg of lithium orotate, approximately 750 mcg of vitamin A (as beta carotene), approximately 60 mg of vitamin C (as ascorbic acid), approximately 5 mcg of vitamin D3 (as cholecalciferol), approximately 2 mg of vitamin B6 (as pyridoxine hydrochloride), approximately 2.4 mcg of vitamin B12 (as cyanocobalamin), approximately 100 mg of choline (as choline bitartrate), approximately 10 mg of vitamin E (as alpha tocopheryl acetate), approximately 500 mcg of vitamin B9 (as L-methyl folate calcium), approximately 300 mg of calcium (as calcium carbonate), approximately 75 mcg of iodine (from kelp), approximately 50 mg of magnesium glycinate, approximately 7.5 mg of zinc (as zinc citrate), approximately 12.5 mcg of selenium (as selenium yeast), approximately 9 mg of iron (as ferrous fumarate), approximately 80 mg of potassium (as potassium citrate), approximately 250 mg of EPA/DHA, and approximately 100 mcg of vitamin K2 (as MK-7). In some cases, the composition may be provided in a two-capsule daily serving format using size 0 or size 1 capsules.

As one alternative example, the composition or compound may include between approximately 4.5 mg and 5.5 mg of lithium orotate, between approximately 675 mcg and 825 mcg of vitamin A (as beta carotene), between approximately 54 mg and 66 mg of vitamin C (as ascorbic acid), between approximately 4.5 mcg and 5.5 mcg of vitamin D3 (as cholecalciferol), between approximately 1.8 mg and 2.2 mg of vitamin B6 (as pyridoxine hydrochloride), between approximately 2.16 mcg and 2.64 mcg of vitamin B12 (as cyanocobalamin), between approximately 90 mg and 110 mg of choline (as choline bitartrate), between approximately 9 mg and 11 mg of vitamin E (as alpha tocopheryl acetate), between approximately 450 mcg and 550 mcg of vitamin B9 (as L-methyl folate calcium), between approximately 270 mg and 330 mg of calcium (as calcium carbonate), between approximately 67.5 mcg and 82.5 mcg of iodine (from kelp), between approximately 45 mg and 55 mg of magnesium glycinate, between approximately 6.75 mg and 8.25 mg of zinc (as zinc citrate), between approximately 11.25 mcg and 13.75 mcg of selenium (as selenium yeast), between approximately 8.1 mg and 9.9 mg of iron (as ferrous fumarate), between approximately 72 mg and 88 mg of potassium (as potassium citrate), between approximately 225 mg and 275 mg of EPA/DHA, and between approximately 90 mcg and 110 mcg of vitamin K2 (as MK-7).

As another example, the composition or compound may include between approximately 4 mg and 6 mg of lithium orotate, between approximately 600 mcg and 900 mcg of vitamin A (as beta carotene), between approximately 48 mg and 72 mg of vitamin C (as ascorbic acid), between approximately 4 mcg and 6 mcg of vitamin D3 (as cholecalciferol), between approximately 1.6 mg and 2.4 mg of vitamin B6 (as pyridoxine hydrochloride), between approximately 1.92 mcg and 2.88 mcg of vitamin B12 (as cyanocobalamin), between approximately 80 mg and 120 mg of choline (as choline bitartrate), between approximately 8 mg and 12 mg of vitamin E (as alpha tocopheryl acetate), between approximately 400 mcg and 600 mcg of vitamin B9 (as L-methyl folate calcium), between approximately 240 mg and 360 mg of calcium (as calcium carbonate), between approximately 60 mcg and 90 mcg of iodine (from kelp), between approximately 40 mg and 60 mg of magnesium glycinate, between approximately 6 mg and 9 mg of zinc (as zinc citrate), between approximately 10 mcg and 15 mcg of selenium (as selenium yeast), between approximately 7.2 mg and 10.8 mg of iron (as ferrous fumarate), between approximately 64 mg and 96 mg of potassium (as potassium citrate), between approximately 200 mg and 300 mg of EPA/DHA, and between approximately 80 mcg and 120 mcg of vitamin K2 (as MK-7).

As a further example, the composition or compound may include between approximately 3 mg and 7 mg of lithium orotate, between approximately 450 mcg and 1050 mcg of vitamin A (as beta carotene), between approximately 36 mg and 84 mg of vitamin C (as ascorbic acid), between approximately 3 mcg and 7 mcg of vitamin D3 (as cholecalciferol), between approximately 1.2 mg and 2.8 mg of vitamin B6 (as pyridoxine hydrochloride), between approximately 1.44 mcg and 3.36 mcg of vitamin B12 (as cyanocobalamin), between approximately 60 mg and 140 mg of choline (as choline bitartrate), between approximately 6 mg and 14 mg of vitamin E (as alpha tocopheryl acetate), between approximately 300 mcg and 700 mcg of vitamin B9 (as L-methyl folate calcium), between approximately 180 mg and 420 mg of calcium (as calcium carbonate), between approximately 45 mcg and 105 mcg of iodine (from kelp), between approximately 30 mg and 70 mg of magnesium glycinate, between approximately 4.5 mg and 10.5 mg of zinc (as zinc citrate), between approximately 7.5 mcg and 17.5 mcg of selenium (as selenium yeast), between approximately 5.4 mg and 12.6 mg of iron (as ferrous fumarate), between approximately 48 mg and 112 mg of potassium (as potassium citrate), between approximately 150 mg and 350 mg of EPA/DHA, and between approximately 60 mcg and 140 mcg of vitamin K2 (as MK-7).

In various examples, the lithium included in the composition or compound may be provided in the form of a lithium compound or a pharmaceutically acceptable salt thereof. For instance, the lithium may be selected from lithium chloride, lithium bromide, lithium carbonate, lithium nitrate, lithium sulfate, lithium acetate, lithium lactate, lithium citrate, lithium aspartate, lithium gluconate, lithium malate, lithium ascorbate, lithium orotate, lithium succinate, and combinations thereof. In some cases, lithium orotate may be preferred due to its bioavailability and tolerability at low doses. In some cases, the quantity or amount of each element, nutritional additive, and/or mineral included in the composition or compound may be derived from quantities and/or amounts typically found in groundwater supplies and soil analyses together with aggerated or expected consumption levels.

In some aspects, the compositions or compounds, discussed herein, may include lithium orotate in an amount between approximately 1 mg and 20 mg. The specific amount of lithium orotate may be selected based on the intended use of the composition, the target population, and the desired level of mood stabilization or cognitive support. For instance, lower amounts of lithium orotate (such as between approximately 1 mg and 5 mg) may be suitable for general nutritional supplementation, while higher amounts (such as between approximately 10 mg and 20 mg) may be appropriate for individuals requiring enhanced mood support as determined by a medical professional.

In some aspects, the composition or compound may include lithium orotate in specific ratios relative to other ingredients. For instance, the ratio of lithium orotate to calcium may be approximately 1:240, the ratio of lithium orotate to potassium may be approximately 1:700, the ratio of lithium orotate to EPA and DHA may be approximately 1:400, the ratio of lithium orotate to magnesium may be approximately 1:84, the ratio of lithium orotate to folate may be approximately 1:80, the ratio of lithium orotate to vitamin C may be approximately 1:18, the ratio of lithium orotate to vitamin E may be approximately 1:4.5, and the ratio of lithium orotate to zinc may be approximately 1:2.2. In some cases, lithium may be present in greater amounts than vitamin B12, vitamin D3, selenium, iodine, and vitamin A. For instance, the ratio of lithium to vitamin B12 may be approximately 2083:1, the ratio of lithium to vitamin D3 may be approximately 250:1, the ratio of lithium to selenium may be approximately 91:1, the ratio of lithium to iodine may be approximately 33:1, and the ratio of lithium to vitamin A may be approximately 5.6:1.

In some aspects, the ratio of lithium orotate to the total of all other ingredients may be approximately 1:1529. In some cases, lithium orotate may represent approximately 0.065% of the total composition by weight. In some aspects, the compositions or compounds may include lithium orotate in an amount between approximately 1 mg and 20 mg, and the ratio of lithium orotate to the total of all other ingredients may range from approximately 1:7645 (at 1 mg lithium orotate) to approximately 1:382 (at 20 mg lithium orotate). In such cases, lithium orotate may represent approximately 0.013% to 0.261% of the total composition by weight.

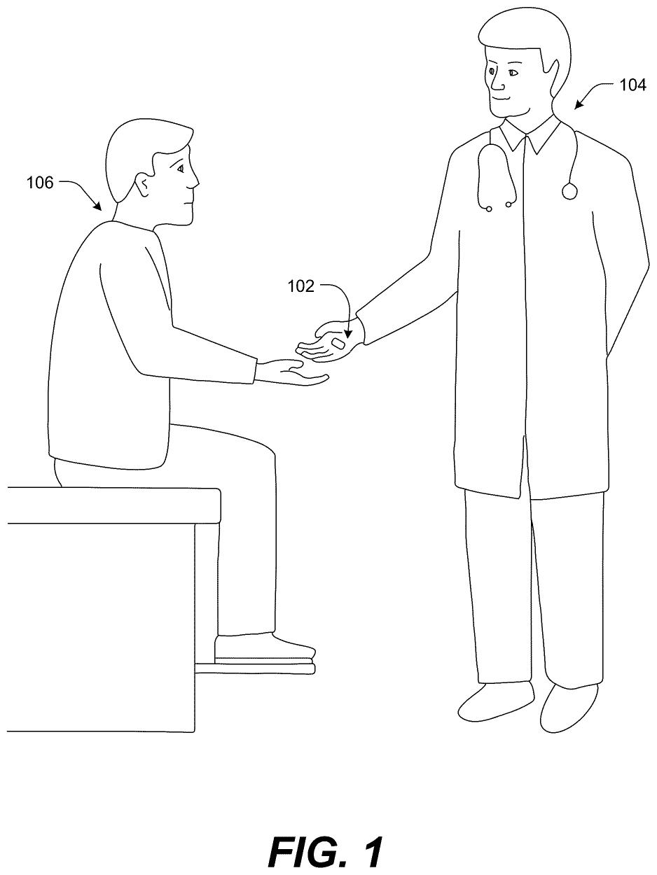

FIG. 1 shows a method of treating a health condition through administration a compound 102 including lithium orotate by medical professional 104. The administration may be in various forms as described below, including tablet or pill, such as shown in the illustrated example. The compound 102 may include lithium orotate (such as in the amount of 5 mg). The compound 102 may also contain various elements or ingredients, such as those discussed above, in various amounts. In some cases, the compound 102 may be formulated to support cognitive and mental health stabilization, mood regulation, reward signaling support, craving balance, calm focus, and metabolic stability in individuals of various demographics, moods, and overall mental well-being.

The therapeutically effective dose of the compounds 102 providing lithium orotate may vary between individual patients 106, in some cases. Initially, the compounds 102 may contain approximately 5 mg of lithium orotate. In some instances, the dosage may be adjusted based on individual patient needs, health conditions, and response to treatment as determined by the medical professional 104. The compound 102 may be administered on a periodic basis, such as daily or weekly, depending on the treatment protocol established by the medical professional 104.

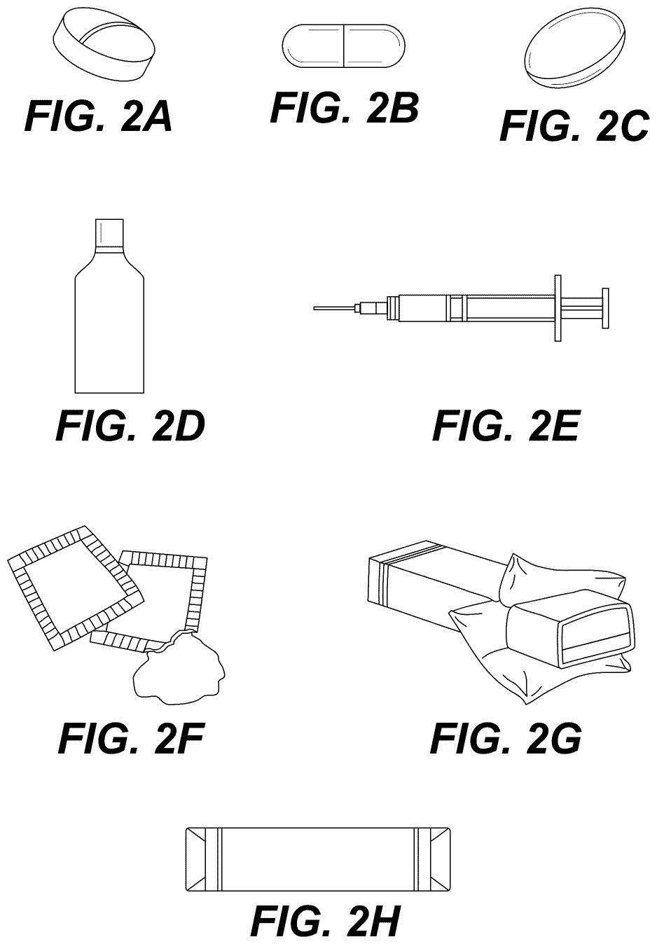

The method of administration of the compound 102 may vary and include various delivery vehicles, such as shown in FIGS. 2A-2H. The compound 102 can be administered by mouth using pills or tablets as shown in FIG. 2A, a two-piece gelatin capsules as shown in FIG. 2B, soft gelcaps as shown in FIG. 2C, or a liquid as shown in FIG. 2D. The compound 102 may also be administered in injectable form as shown in FIG. 2E. In addition, the compound 102 may be administered by adding them to food in either a liquid or powder form, as shown in FIGS. 2D and 2F. The compound 102 may also be administered through prepared foods as shown in FIG. 2G or individually wrapped sticks of chewing gum shown in FIG. 2H. In some cases, the composition may be provided in a two-capsule daily serving format using vegetable capsules. A practitioner of ordinary skill in the art would understand that a variety of commonly available forms similar to those shown in FIGS. 2A-2H could also be used to administer the compounds 102, such as via a drink or beverage form. Accordingly, a practitioner of ordinary skill in the art would understand that a variety of commonly available forms similar to those shown in FIGS. 2A-2H could also be used to administer lithium compounds or a combined lithium/cannabinoid medication as discussed below

In some aspects, the composition or compound may be formulated for reward signaling support, craving balance, calm focus, and metabolic stability. For instance, the composition or compound may include approximately 5 mg of lithium (elemental from orotate), approximately 600 mg of palmitoylethanolamide (PEA), approximately 500 mg of DL-phenylalanine (DLPA), approximately 200 mg of L-theanine, approximately 120 mg of magnesium (elemental from glycinate chelate), approximately 300 mg of ashwagandha extract, approximately 300 mg of dihydroberberine (DHB), approximately 500 mg of myo-inositol, and approximately 200 mcg of chromium (as chromium picolinate). In some cases, the composition may be provided in a two-capsule daily serving format using vegetable capsules. The palmitoylethanolamide (PEA) may support endocannabinoid system function and may in some aspects assist with modulating reward signaling pathways and reducing unwanted cravings. The DL-phenylalanine (DLPA) may support dopamine synthesis and may in some cases assist with promoting dopamine balance and emotional steadiness. The L-theanine may support calm focus and relaxation without sedation, which may in some aspects assist with impulse control and stress-related urges. The ashwagandha extract may support stress response through modulation of cortisol levels and may in some instances assist with emotional steadiness and managing stress-related urges. The dihydroberberine (DHB) may support metabolic function and glucose regulation. The myo-inositol may support cellular signaling and mood regulation. The chromium picolinate may support healthy blood sugar levels and metabolic stability.

As one alternative example, the composition or compound may include between approximately 4.5 mg and 5.5 mg of lithium (elemental from orotate), between approximately 540 mg and 660 mg of palmitoylethanolamide (PEA), between approximately 450 mg and 550 mg of DL-phenylalanine (DLPA), between approximately 180 mg and 220 mg of L-theanine, between approximately 108 mg and 132 mg of magnesium (elemental from glycinate chelate), between approximately 270 mg and 330 mg of ashwagandha extract, between approximately 270 mg and 330 mg of dihydroberberine (DHB), between approximately 450 mg and 550 mg of myo-inositol, and between approximately 180 mcg and 220 mcg of chromium (as chromium picolinate).

As another example, the composition or compound may include between approximately 4 mg and 6 mg of lithium (elemental from orotate), between approximately 480 mg and 720 mg of palmitoylethanolamide (PEA), between approximately 400 mg and 600 mg of DL-phenylalanine (DLPA), between approximately 160 mg and 240 mg of L-theanine, between approximately 96 mg and 144 mg of magnesium (elemental from glycinate chelate), between approximately 240 mg and 360 mg of ashwagandha extract, between approximately 240 mg and 360 mg of dihydroberberine (DHB), between approximately 400 mg and 600 mg of myo-inositol, and between approximately 160 mcg and 240 mcg of chromium (as chromium picolinate).

As a further example, the composition or compound may include between approximately 3 mg and 7 mg of lithium (elemental from orotate), between approximately 360 mg and 840 mg of palmitoylethanolamide (PEA), between approximately 300 mg and 700 mg of DL-phenylalanine (DLPA), between approximately 120 mg and 280 mg of L-theanine, between approximately 72 mg and 168 mg of magnesium (elemental from glycinate chelate), between approximately 180 mg and 420 mg of ashwagandha extract, between approximately 180 mg and 420 mg of dihydroberberine (DHB), between approximately 300 mg and 700 mg of myo-inositol, and between approximately 120 mcg and 280 mcg of chromium (as chromium picolinate).

In some aspects, the composition or compound may be provided in a capsule form with a serving size of two capsules. For instance, the composition or compound may include approximately 5 mg of lithium (elemental from lithium orotate), approximately 600 mg of palmitoylethanolamide (PEA), approximately 500 mg of DL-phenylalanine (DLPA), approximately 200 mg of L-theanine, approximately 120 mg of magnesium (elemental from glycinate chelate), approximately 300 mg of ashwagandha extract, approximately 300 mg of dihydroberberine (DHB), approximately 500 mg of myo-inositol, and approximately 200 mcg of chromium (as chromium picolinate), along with other ingredients such as microcrystalline cellulose or rice flour, silicon dioxide, and vegetable magnesium stearate. The capsules may be vegetable capsules (hypromellose) and may be size 00 capsules that are clear or white in color. The composition may be configured for administration of two capsules once daily with food or as directed by a healthcare professional.

Although the discussion above sets forth example implementations of the described techniques, other architectures may be used to implement the described functionality and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A composition comprising:
between approximately 270 mg and 1320 mg of calcium;
between approximately 67.5 mcg and 165 mcg of iodine as potassium iodide;
between approximately 45 mg and 462 mg of magnesium;
between approximately 6.75 mg and 33 mg of zinc;
between approximately 11.25 mcg and 60.5 mcg of selenium;
between approximately 4.0 mg and 11 mg of lithium;
between approximately 72 mg and 3.85 g of potassium;
between approximately 225 mg and 2100 mg of eicosapentaenoic acid (EPA) and docosahexaenoic acid (DHA);
between approximately 4.5 mcg and 22 mcg of vitamin D3 as cholecalciferol;
between approximately 1.7 mcg and 2.7 mcg of vitamin B12 as cyanocobalamin;
between approximately 510 mcg and 990 mcg of vitamin A as beta carotene;
between approximately 54 mg and 100 mg of vitamin C as ascorbic acid;
between approximately 9 mg and 25 mg of vitamin E; and
between approximately 250 mcg and 550 mcg of folate.

2. The composition of claim 1, wherein the composition comprises:
approximately 1200 mg of calcium;
approximately 150 mcg of iodine as potassium iodide;
approximately 420 mg of magnesium;
approximately 30 mg of zinc as zinc biglycinate;
approximately 55 mcg of selenium;
approximately 5 mg of lithium as lithium orotate;
approximately 3.5 g of potassium;
approximately 1000 mg of eicosapentaenoic acid (EPA) and docosahexaenoic acid (DHA);
approximately 20 mcg of vitamin D3 as cholecalciferol;
approximately 2.4 mcg of vitamin B12 as cyanocobalamin;
approximately 900 mcg of vitamin A as beta carotene;
approximately 90 mg of vitamin C as ascorbic acid;
approximately 22.5 mg of vitamin E as di-alpha tocopheryl acetate; and
approximately 400 mcg of folate.

3. The composition of claim 1, further comprising:
between approximately 108 mcg and 132 mcg of vitamin K;
between approximately 1.08 mg and 1.32 mg of thiamin;
between approximately 1.17 mg and 1.43 mg of riboflavin;
between approximately 14.4 mg and 17.6 mg of niacin;
between approximately 1.17 mg and 1.43 mg of vitamin B6 as pyridoxine hydrochloride;
between approximately 27 mcg and 33 mcg of biotin;
between approximately 9 mg and 11 mg of pantothenic acid as d-calcium pantothenate;
between approximately 630 mg and 770 mg of phosphorus;
between approximately 0.81 mg and 0.99 mg of copper;
between approximately 450 mg and 550 mg of choline;
between approximately 2.07 mg and 2.53 mg of manganese;
between approximately 31.5 mcg and 38.5 mcg of chromium;
between approximately 2.07 g and 2.53 g of chloride;
between approximately 13.5 mg and 16.5 mg of lutein; and
between approximately 18 mg and 22 mg of lycopene.

4. The composition of claim 1, wherein the composition is formulated for pediatric use and comprises:
approximately 600 mcg of vitamin A as beta carotene;
approximately 75 mg of vitamin C as ascorbic acid;
approximately 600 IU of vitamin D3 as cholecalciferol;
approximately 12 mg of vitamin E as di-alpha tocopheryl acetate;
approximately 60 mcg of vitamin K;
approximately 0.9 mg of thiamin;
approximately 0.9 mg of riboflavin;
approximately 12 mg of niacin;
approximately 1 mg of vitamin B6 as pyridoxine hydrochloride;
approximately 300 mcg of folate as dietary folate equivalents;
approximately 1.8 mcg of vitamin B12 as cyanocobalamin;
approximately 20 mcg of biotin;
approximately 4 mg of pantothenic acid as d-calcium pantothenate;
approximately 1300 mg of calcium;
approximately 1250 mg of phosphorus;
approximately 120 mcg of iodine as potassium iodide;
approximately 240 mg of magnesium;
approximately 11 mg of zinc as zinc biglycinate;
approximately 8 mg of iron as ferrous fumarate;
approximately 40 mcg of selenium;
approximately 700 mcg of copper;
approximately 1.8 mg of manganese;
approximately 5 mg of lithium as lithium orotate;
approximately 24 mcg of chromium;
approximately 2.3 g of chloride;
approximately 2.4 g of potassium;
approximately 6 mg of lutein; and
approximately 2000 mg of EPA and DHA.

5. The composition of claim 1, wherein the composition comprises:
approximately 750 mcg of vitamin A as beta carotene;
approximately 60 mg of vitamin C as ascorbic acid;
approximately 5 mcg of vitamin D3 as cholecalciferol;
approximately 2 mg of vitamin B6 as pyridoxine hydrochloride;
approximately 2.4 mcg of vitamin B12 as cyanocobalamin;
approximately 100 mg of choline as choline bitartrate;
approximately 10 mg of vitamin E as alpha tocopheryl acetate;
approximately 500 mcg of vitamin B9 as L-methyl folate calcium;
approximately 300 mg of calcium as calcium carbonate;
approximately 75 mcg of iodine;
approximately 50 mg of magnesium glycinate;
approximately 7.5 mg of zinc as zinc citrate;
approximately 12.5 mcg of selenium as selenium yeast;
approximately 9 mg of iron as ferrous fumarate;
approximately 80 mg of potassium as potassium citrate;
approximately 5 mg of lithium as lithium orotate;
approximately 250 mg of EPA and DHA; and
approximately 100 mcg of vitamin K2 as MK-7.

6. The composition of claim 1, wherein the zinc is provided as zinc biglycinate, the lithium is provided as lithium orotate, and the vitamin E is provided as di-alpha tocopheryl acetate.

7. The composition of claim 1, wherein the lithium is present in an amount between approximately 4.5 mg and 5.5 mg.

8. The composition of claim 1, wherein the eicosapentaenoic acid (EPA) and the docosahexaenoic acid (DHA) are present in a ratio of approximately 2:1.

9. A composition comprising:
between approximately 4.0 mg and 12 mg of lithium orotate;
between approximately 45 mg and 462 mg of magnesium; and
between approximately 225 mg and 2100 mg of eicosapentaenoic acid (EPA) and docosahexaenoic acid (DHA) present in a ratio of approximately 2:1.

10. The composition of claim 9, further comprising:
between approximately 270 mg and 1320 mg of calcium;
between approximately 67.5 mcg and 165 mcg of iodine as potassium iodide;
between approximately 6.75 mg and 33 mg of zinc;
between approximately 11.25 mcg and 60.5 mcg of selenium;
between approximately 72 mg and 3.85 g of potassium;
between approximately 4.5 mcg and 22 mcg of vitamin D3 as cholecalciferol;
between approximately 1.7 mcg and 2.7 mcg of vitamin B12 as cyanocobalamin;
between approximately 510 mcg and 990 mcg of vitamin A as beta carotene;
between approximately 54 mg and 100 mg of vitamin C as ascorbic acid;
between approximately 9 mg and 25 mg of vitamin E; and
between approximately 250 mcg and 550 mcg of folate.

11. The composition of claim 9, further comprising:
approximately 1200 mg of calcium;
approximately 150 mcg of iodide;
approximately 420 mg of magnesium;
approximately 30 mg of zinc biglycinate;
approximately 55 mcg of selenium;
approximately 5.0 mg of lithium orotate;
approximately 3.5 g of potassium;
approximately 1000+1000 mg of EPA and DHA in a ratio of 2:1;
approximately 20 mcg of vitamin D3 as cholecalciferol;
approximately 2.4 mcg of vitamin B12 as cyanocobalamin;
approximately 900 mcg of vitamin A;
approximately 90 mg of vitamin C;
approximately 22.5 mg of vitamin E; and
approximately 400 mcg of folate.

12. The composition of claim 9, further comprising:
between approximately 108 mcg and 132 mcg of vitamin K;
between approximately 1.08 mg and 1.32 mg of thiamin;
between approximately 1.17 mg and 1.43 mg of riboflavin;
between approximately 14.4 mg and 17.6 mg of niacin;
between approximately 1.17 mg and 1.43 mg of vitamin B6 as pyridoxine hydrochloride;
between approximately 27 mcg and 33 mcg of biotin;
between approximately 9 mg and 11 mg of pantothenic acid as d-calcium pantothenate;
between approximately 630 mg and 770 mg of phosphorus;
between approximately 0.81 mg and 0.99 mg of copper;
between approximately 450 mg and 550 mg of choline;
between approximately 2.07 mg and 2.53 mg of manganese;
between approximately 31.5 mcg and 38.5 mcg of chromium;
between approximately 2.07 g and 2.53 g of chloride;
between approximately 13.5 mg and 16.5 mg of lutein; and
between approximately 18 mg and 22 mg of lycopene.

13. The composition of claim 9, wherein the composition is formulated for pediatric use and comprises:
approximately 600 mcg of vitamin A as beta carotene;
approximately 75 mg of vitamin C as ascorbic acid;
approximately 600 IU of vitamin D3 as cholecalciferol;
approximately 12 mg of vitamin E as di-alpha tocopheryl acetate;
approximately 60 mcg of vitamin K;
approximately 0.9 mg of thiamin;
approximately 0.9 mg of riboflavin;
approximately 12 mg of niacin;
approximately 1 mg of vitamin B6 as pyridoxine hydrochloride;
approximately 300 mcg of folate as dietary folate equivalents;
approximately 1.8 mcg of vitamin B12 as cyanocobalamin;
approximately 20 mcg of biotin;
approximately 4 mg of pantothenic acid as d-calcium pantothenate;
approximately 1300 mg of calcium;
approximately 1250 mg of phosphorus;
approximately 120 mcg of iodine as potassium iodide;
approximately 240 mg of magnesium;
approximately 11 mg of zinc as zinc biglycinate;
approximately 8 mg of iron as ferrous fumarate;
approximately 40 mcg of selenium;
approximately 700 mcg of copper;
approximately 1.8 mg of manganese;
approximately 5 mg of lithium as lithium orotate;
approximately 24 mcg of chromium;
approximately 2.3 g of chloride;
approximately 2.4 g of potassium;
approximately 6 mg of lutein; and
approximately 2000 mg of EPA and DHA.

14. A composition comprising:
between approximately 1.0 mg and 12 mg of lithium orotate;
between approximately 45 mg and 462 mg of magnesium; and
between approximately 225 mg and 2100 mg of L-theanine.

15. The composition of claim 14, further comprising:
between approximately 270 mg and 660 mg of palmitoylethanolamide (PEA);
between approximately 225 mg and 550 mg of DL-phenylalanine (DLPA);
between approximately 135 mg and 330 mg of ashwagandha extract;
between approximately 135 mg and 330 mg of dihydroberberine (DHB);
between approximately 225 mg and 550 mg of myo-inositol; and
between approximately 90 mcg and 220 mcg of chromium as chromium picolinate.

16. The composition of claim 14, further comprising:
between approximately 90 mg and 110 mg of magnesium as magnesium glycinate;
between approximately 7.2 mg and 8.8 mg of zinc as zinc bisglycinate chelate;
between approximately 90 mcg and 110 mcg of selenium as selenomethionine;
between approximately 5.4 mg and 6.6 mg of astaxanthin;
between approximately 67.5 mg and 82.5 mg of coenzyme Q10; and
between approximately 90 mg and 110 mg of alpha-lipoic acid.

17. The composition of claim 14, further comprising:
between approximately 120 mg and 280 mg of Vitex agnus-castus extract;
between approximately 18 mg and 42 mg of saffron stigma extract;
between approximately 150 mg and 350 mg of ginger root extract;
between approximately 30 mg and 70 mg of curcumin extract;
between approximately 3 mg and 7 mg of black pepper fruit extract;
between approximately 180 mg and 420 mg of magnesium bisglycinate chelate;
between approximately 12 mg and 28 mg of vitamin B6 as pyridoxal-5-phosphate; and
between approximately 120 mg and 280 mg of myo-inositol.

18. The composition of claim 17, further comprising:
approximately 5 mg of lithium as lithium orotate;
approximately 200 mg of Vitex agnus-castus extract;
approximately 30 mg of saffron stigma extract;
approximately 250 mg of ginger root extract;
approximately 50 mg of curcumin extract;
approximately 5 mg of black pepper fruit extract;
approximately 200 mg of L-theanine;
approximately 300 mg of magnesium bisglycinate chelate;
approximately 20 mg of vitamin B6 as pyridoxal-5-phosphate; and
approximately 200 mg of myo-inositol.

19. The composition of claim 14, further comprising between approximately 225 mg and 2100 mg of eicosapentaenoic acid (EPA) and docosahexaenoic acid (DHA) in a 2:1 ratio.

20. The composition of claim 14, further comprising:
approximately 750 mcg of vitamin A as beta carotene;
approximately 60 mg of vitamin C as ascorbic acid;
approximately 5 mcg of vitamin D3 as cholecalciferol;
approximately 2 mg of vitamin B6 as pyridoxine hydrochloride;
approximately 2.4 mcg of vitamin B12 as cyanocobalamin;
approximately 100 mg of choline as choline bitartrate;
approximately 10 mg of vitamin E as alpha tocopheryl acetate;
approximately 500 mcg of vitamin B9 as L-methyl folate calcium;
approximately 300 mg of calcium as calcium carbonate;
approximately 75 mcg of iodine;
approximately 50 mg of magnesium glycinate;
approximately 7.5 mg of zinc as zinc citrate;
approximately 12.5 mcg of selenium as selenium yeast;
approximately 9 mg of iron as ferrous fumarate;
approximately 80 mg of potassium as potassium citrate;
approximately 5 mg of lithium as lithium orotate;
approximately 250 mg of EPA and DHA; and
approximately 100 mcg of vitamin K2 as MK-7.
